(19) United States
(12) Patent Application Publication
(10) Pub. No.: US 2022/0071499 A1
(43) Pub. Date: Mar. 10, 2022

(54) INTRAOPERATIVE BLOOD FLOW IMAGING METHOD BASED ON FLUORESCENCE IMAGING

(71) Applicant: Nanjing Nuoyuan Medical Devices Co., Ltd., Nanjing (CN)

(72) Inventors: Huiming Cai, Nanjing (CN); Changliu Li, Nanjing (CN); Kena Ni, Nanjing (CN)

(21) Appl. No.: 17/394,161

(22) Filed: Aug. 4, 2021

(30) Foreign Application Priority Data
Sep. 9, 2020 (CN) 202010941608.X

(51) Int. Cl. A61B 5/026; G06T 5/40; G06T 5/00; G06T 5/20; G06T 7/11; G06T 7/155

(52) U.S. Cl. CPC A61B 5/0261; G06T 5/40; G06T 5/002; A61B 5/0071; G06T 7/11; G06T 7/155; G06T 5/20

(57) ABSTRACT

The embodiment of the invention discloses an intraoperative blood flow imaging method based on fluorescence imaging and belongs to the technical field of medical fluorescence imaging. The present invention effectively removes shadow or tissue pulsation interference by utilizing the fluorescence imaging images to identify the blood flow area; and meanwhile, by conducting pseudo-color imaging on the blood flow area, as a pseudo-color value has correlation with a blood flow perfusion time, the present invention may accurately indicate the blood flow areas in the images and is easy to distinguish the blood flow areas.

### CROSS-REFERENCE TO RELATED APPLICATIONS

This application claims priority to Chinese Patent Application No. 202010941608.X with a filing date of Sep. 9, 2020. The content of the aforementioned applications, including any intervening amendments thereto, are incorporated herein by reference.

### TECHNICAL FIELD

The embodiment of the invention relates to the technical field of medical fluorescence imaging, in particular to an intraoperative blood flow imaging method based on fluorescence imaging.

### BACKGROUND

Clinic blood flow perfusion information has important significance for clinically diagnosing diseases. Common technical means mostly adopt a method of iconography, such as a method based on magnetic resonance, CT and ultrasonic images.

However, the conventional method of iconography is basically unavailable in an operation as existing intraoperative blood perfusion assessment is limited to an operation time, so that an effective intraoperative blood flow imaging method is urgently needed to help doctors to make effective and accurate diagnosis.

Based on the above, the present invention provides an intraoperative blood flow imaging method based on fluorescence imaging to solve the above problems.

### SUMMARY

The embodiment of the invention provides an intraoperative blood flow imaging method based on fluorescence imaging and aims at solving the technical problems mentioned in the background.

The embodiment of the invention provides the intraoperative blood flow imaging method based on fluorescence imaging. In one feasible solution, the method comprises the following steps of:

S1, setting a starting time t1 and an ending time t2 of fluorescence imaging observation in an operative area, and obtaining an image sequence in a time period t1-t2;

S2, preprocessing the image sequence to obtain a to-be-identified image sequence Itp of a blood flow;

S3, obtaining a pixel maximum value ptx of a frame with the maximum brightness in a blood flow area in an identified image through a blood flow area identification algorithm on the Itp image sequence;

S4, extracting a pixel gray value of the blood flow area at the time of t1-t2, conducting pseudo-color space mapping, and obtaining an image It after pseudo-color mapping at the time t; and

S5, obtaining a sum I of the images of the pseudo-color image It at each time in the time period of t1-t2, and normalizing the gray values in I to obtain a final blood flow image.

The embodiment of the invention provides the intraoperative blood flow imaging method based on fluorescence imaging. In one feasible solution, the preprocessing in S2 comprises the following step of:

enhancing a gray level of the image sequence by adopting a self-adaptive histogram algorithm, and conducting noise filtering processing on the enhanced image sequence to obtain a to-be-identified image sequence Itp of the blood flow.

The embodiment of the invention provides the intraoperative blood flow imaging method based on fluorescence imaging. In one feasible solution, the noise filtering processing algorithm is a guide filtering algorithm.

The embodiment of the invention provides the intraoperative blood flow imaging method based on fluorescence imaging. In one feasible solution, the blood flow region identification algorithm in S3 comprises the following steps of:

searching the frame with the maximum brightness in the Itp image sequence at t1-t2, calculating a pixel of each frame for equalization processing, and calculating a maximum average value at the time tx as the frame with the maximum brightness in the image sequence; and calculating the pixel maximum value ptx in the frame with the maximum brightness at the time tx, segmenting the corresponding image with a pixel maximum value point as a seed point to obtain a segmented area as the blood flow area in the image.

The embodiment of the invention provides the intraoperative blood flow imaging method based on fluorescence imaging. In one feasible solution, the image segmentation algorithm is a watershed algorithm.

The embodiment of the invention provides the intraoperative blood flow imaging method based on fluorescence imaging. In one feasible solution, when t<t1, an image gray map color value P=(ptx (t1−t)/255, 0, 0); and

when t>t1, the image gray map color value P=(0, 0, ptx (t41)/255).

Based on the solution, the present invention has the beneficial effects that shadow or tissue pulsation interference is effectively removed by utilizing the fluorescence imaging images to identify the blood flow area; and meanwhile, by conducting pseudo-color imaging on the blood flow area, as a pseudo-color value has correlation with a blood flow perfusion time, the present invention may accurately indicate the blood flow areas in the images and is easy to distinguish the blood flow areas. The embodiment of the invention discloses an intraoperative blood flow imaging method based on fluorescence imaging and belongs to the technical field of medical fluorescence imaging. The intraoperative blood flow imaging method based on fluorescence imaging comprises the following steps of S1, setting a starting time t1 and an ending time t2 of fluorescence imaging observation in an operative area to obtain an image sequence in a time period t1-t2; S2, preprocessing the image sequence to obtain a to-be-identified image sequence Itp of a blood flow; S3, obtaining a pixel maximum value ptx of a frame with the maximum brightness in a blood flow area in an identified image through a blood flow area identification algorithm on the Itp image sequence; and S4, extracting the pixel gray value of the blood flow area at the time t1-t2, conducting pseudo-color space mapping, and obtaining an image It after pseudo-color mapping at the time t. The present invention effectively removes shadow or tissue pulsation interference by utilizing the fluorescence imaging images to identify the blood flow area; and meanwhile, by conducting pseudo-color imaging on the blood flow area, as a pseudo-color value has correlation with a blood flow perfusion time, the present invention may accurately indicate the blood flow areas in the images and is easy to distinguish the blood flow areas.

### BRIEF DESCRIPTION OF DRAWINGS

In order to more clearly illustrate the embodiments of the present invention or the technical solution in the prior art, the drawings used in the embodiments or the description of the prior art will be briefly described below, and it is obvious that the drawings in the following description are some embodiments of the present invention, and those skilled in the art can obtain other drawings based on the drawings without inventive labor.

FIG. 1 is a block flow diagram of an imaging method of the present invention.

### DETAILED DESCRIPTION

In order to make objectives, technical schemes and advantages of the present invention more clear, the technical solutions in the embodiments of the present invention are clearly and completely described below in combination with drawings in the embodiments of the present invention. Obviously, the embodiments described are only part of the embodiments rather than all the embodiments. Based on the embodiments of the present invention, all the other embodiments obtained by those of ordinary skill without any creative works are within the protection scope of the present invention.

In the description of the present invention, it is to be understood that orientations and positional relationships indicated by the terms “central”, “longitudinal”, “lateral”, “upper”, “lower”, “front”, “rear”, “left”, “right”, “vertical”, “horizontal”, “top”, “bottom”, “inner”, “outer”, “axial”, “radial”, “circumferential” and the like are based on the orientation or positional relationship illustrated in the drawings for convenience in describing the present invention and to simplify the description, and are not intended to indicate or imply that the indicated device or element must have a particular orientation, be constructed and operated in a particular orientation, and thus are not to be construed as limiting the invention.

In the present invention, unless otherwise specifically stated or limited, the terms “mounted”, “connected”, “fixed” and the like are to be construed broadly and may include, for example, fixed, detachable or integral connections, mechanical connection, electrical connection, communication connection, direct connection or indirect connection through an intervening medium, communication between two elements or interaction between two elements, unless otherwise specifically stated or limited. The specific meanings of the above terms in the present invention can be understood according to specific situations by those of ordinary skill in the art.

The technical solution of the present invention will be described in detail below through specific embodiments. These several specific embodiments may be combined with each other below, and details of the same or similar concepts or processes may not be repeated in some embodiments.

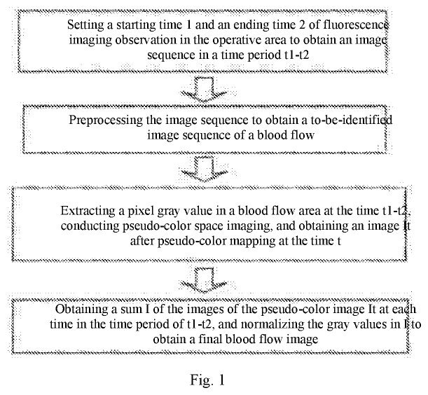

FIG. 1 is a diagram illustrating an intraoperative blood flow imaging method based on fluorescence imaging of the present invention, and the intraoperative blood flow imaging method based on fluorescence imaging comprises the following steps of:

S1, setting a starting time t1 and an ending time t2 of fluorescence imaging observation in an operative area, and obtaining an image sequence in a time period t1-t2;

S2, preprocessing the image sequence to obtain a to-be-identified image sequence Itp of a blood flow;

S3, obtaining a pixel maximum value ptx of a frame with the maximum brightness in a blood flow area in an identified image through a blood flow area identification algorithm on the Itp image sequence;

S4, extracting a pixel gray value of the blood flow area at the time of t1-t2, conducting pseudo-color space mapping, and obtaining an image It after pseudo-color mapping at the time t; and

S5, obtaining a sum I of the images of the pseudo-color image It at each time in the time period of t1-t2, and normalizing the gray values in Ito obtain a final blood flow image.

It is easy to find from the above contents that by using the intraoperative blood flow imaging method based on fluorescence imaging of the present invention, intraoperative blood flow imaging images may be obtained; by recording the starting time t1 and the ending time t2 of fluorescence imaging observation in the operative area and obtaining the image sequence in the time period of t1-t2, so that noise interference is lowered, and the blood vessel characteristics are enhanced through the preprocessing means, such as gray level enhancement and noise filtering, and finally, the to-be-identified image sequence Itp of the blood flow is obtained; by using the blood flow area identification algorithm to identify the to-be-identified image sequence of the blood flow, the pixel maximum value ptx of the frame with the maximum brightness at time tx is obtained; then the pixel gray values in the blood flow area from the time t1 to the time t2 according to the pixel maximum value ptx of the frame with the maximum brightness at the time tx, and the images It after pseudo-color mapping at time t are obtained, then the pseudo-color images are fused, and during fusion, the blood flow images are obtained by obtaining the sum I of the images at each time It in the time period of t1-t2 according to the gray values in I, so that in the process of identifying the blood flow area with such mode, shadow or tissue pulsation interference can be effectively removed to enable the blood flow area to be imaged through pseudo-color imaging, a pseudo-color value has correlation with a blood flow perfusion time, and the present invention has the characteristics of accurately indicating the blood flow areas in the images and being easy to distinguish the blood flow areas.

Alternatively, in this embodiment, the preprocessing in S2 comprises the following steps of:

enhancing the gray level of the image sequence by adopting a self-adaptive histogram algorithm, and conducting noise filtering processing on the enhanced image sequence to obtain a to-be-identified image sequence Itp of a blood flow. It should be noted that, in this embodiment, through preprocessing in this way, the gray level of the image sequence may be enhanced; and by conducting noise filtering on the enhanced gray image sequence, the blood vessel characteristics may be conveniently enhanced, and subsequent blood flow area identification work is facilitated.

In addition, the noise filtering processing algorithm is a guide filtering algorithm; guide filtering is to output a deformed guide filtering image by using a guide image as a filtering content image to achieve local linear function expression on the guide image and achieve various different linear transformations; and as a mean value and a variance of guide filtering computation may be rapidly obtained by searching an integrogram, a speed of guide filtering can be very rapid, and guide filtering, as edge-preserving filtering, has obvious speed advantage compared with bilinear filtering.

More specifically, the blood flow area identification algorithm in S3 comprises the following steps of:

searching the frame with the maximum brightness in the Itp image sequence at t1-t2, calculating a pixel of each frame for equalization processing, and calculating a maximum average value at the time tx as the frame with the maximum brightness in the image sequence; and calculating the pixel maximum value ptx in the frame with the maximum brightness at the time tx, segmenting the corresponding image with a pixel maximum value point as a seed point to obtain a segmented area as the blood flow area in the image, wherein through the blood flow area identification algorithm in such a way, the frame with the maximum brightness in the image sequence at the time tx can be conveniently searched, and the pixel maximum value ptx at the time tx is calculated according to the frame with the brightness maximum, so that the blood flow area in the image is obtained by segmenting the image.

Further, the image segmentation algorithm is a watershed algorithm which has various implementation algorithms, such as topology, morphology, immersion simulation and precipitation simulation. The watershed algorithm considers image segmentation according to composition of a watershed; the algorithm can limit a gradient image by using a threshold value to eliminate excessive segmentation caused by tiny change of the gray values, and an appropriate area is obtained; and then the gray levels of edge points of the areas are sequenced from low to high, and the gradient image is calculated by using a Sobe 1 operator in the process of realizing submergence from low to high.

Preferably, when t<t1, the image gray mapping color value P=(ptx (t1−t)/255, 0, 0); and

when t>t1, an image gray mapping color value P=(0, 0, ptx (t−t1)/255), wherein P represents a pseudo-color value of the point, which is represented in an RGB color space, and a color value is represented by (R, G, B).

In the present invention, unless expressly stated or limited otherwise, that the first feature is “on” or “under” the second feature may be directly contacting the first feature with the second feature, or indirectly in contacting the first feature with the second feature through an intervening medium.

Moreover, that the first feature is “on”, “above” and “over” the second feature may represent that the first feature is right on or obliquely above the second feature, or merely means that the first feature is at a higher level than the second feature. That the first feature is “beneath”, “below” and “beneath” the second feature may represent that the first feature is right below or obliquely beneath the second feature, or merely means that the first feature is at a lower level than the second feature.

In the description of the specification, with reference to the description of “one embodiment”, “some embodiments”, “an example”, “a specific example”, “some examples” or the like is intended to mean that a particular feature, structure, material, or characteristic described in combination with the embodiment or example is included in at least one embodiment or example of the present invention. In this specification, the schematic representations of the terms used above are not necessarily intended to refer to the same embodiment or example. Moreover, the particular feature, structure, material or characteristic described may be combined in any suitable manner in any one or more of the embodiments or examples. Further, various embodiments or examples and the features of various embodiments or examples described in this specification can be combined by one skilled in the art without contradicting each other.

Finally, it should be noted that: the above embodiments are only used to explain the technical solution of the present invention and shall not be construed as limitation. Although the present invention has been described in detail with reference to the foregoing embodiments, it should be understood by those skilled in the art that modifications may still be made to the technical solution described in the foregoing embodiments, or equivalent substitutions are made on a part of or the whole technical features; and these modifications or substitutions do not deviate the nature of the corresponding technical solution from the scope of the technical solution in respective embodiments of the present invention.

We claim:

1. An intraoperative blood flow imaging method based on fluorescence imaging, characterized by comprising the following steps of:
S1, setting a starting time t1 and an ending time t2 of fluorescence imaging observation in an operative area, and obtaining an image sequence in a time period t1-t2;
S2, preprocessing the image sequence to obtain a to-be-identified image sequence Itp of a blood flow;
S3, obtaining a pixel maximum value ptx of a frame with the maximum brightness in a blood flow area in an identified image through a blood flow area identification algorithm on the Itp image sequence;
S4, extracting a pixel gray value of the blood flow area at the time of t1-t2, conducting pseudo-color space mapping, and obtaining an image It after pseudo-color mapping at the time t; and
S5, obtaining a sum I of the images of the pseudo-color image It at each time in the time period of t1-t2, and normalizing the gray values in Ito obtain a final blood flow image.

2. The intraoperative blood flow imaging method based on fluorescence imaging according to claim 1, wherein the preprocessing in S2 comprises the following steps of:
enhancing a gray level of the image sequence by adopting a self-adaptive histogram algorithm, and conducting noise filtering processing on the enhanced image sequence to obtain the to-be-identified image sequence Itp of the blood flow.

3. The intraoperative blood flow imaging method based on fluorescence imaging according to claim 2, wherein the noise filtering processing algorithm is a guide filtering algorithm.

4. The intraoperative blood flow imaging method based on fluorescence imaging according to claim 1, wherein the blood flow area identification algorithm in S3 comprises the following steps of:
searching the frame with the maximum brightness in the Itp image sequence at t1-t2, calculating a pixel of each frame for equalization processing, and calculating a maximum average value at the time tx as the frame with the maximum brightness in the image sequence; and calculating the pixel maximum value ptx in the frame with the maximum brightness at the time tx, segmenting the corresponding image with a pixel maximum value point as a seed point to obtain a segmented area as the blood flow area in the image.

5. The intraoperative blood flow imaging method based on fluorescence imaging according to claim 4, wherein an image segmentation algorithm is a watershed algorithm.

6. The intraoperative blood flow imaging method based on fluorescence imaging according to claim 1, wherein when t<t1, an image gray mapping color value P=(ptx (t1−t)/255, 0, 0); and
when t>t1, the image gray mapping color value P=(0,0, ptx (t−t1)/255).
